(19) United States
(12) Patent Application Publication
(10) Pub. No.: US 2026/0337604 A1
(43) Pub. Date: Oct. 29, 2026

(54) DIRECT LOADING

(71) Applicant: Droplet Genomics, UAB, Vilnius (LT)

(72) Inventors: Karolis LEONAVICIUS, Vilnius (LT); Rapolas ZILIONIS, Vilnius (LT); Andrew Robert WATSON, Denver, CO (US); Juozas NAINYS, Vilnius (LT); Vaidotas KISELIOVAS, Vilnius (LT)

(21) Appl. No.: 19/486,467

(22) PCT Filed: May 24, 2024
(86) PCT No.: PCT/EP2024/064397

§ 371 Date: Nov. 21, 2025

Related U.S. Application Data
(60) Provisional application No. 63/504,730, filed on May 27, 2023.

(51) Int. Cl. C12N 15/10

(52) U.S. Cl. CPC C12N 15/1093

(57) ABSTRACT

Approaches for direct loading of sample subdivisions onto assay surfaces are disclosed. Through practice of the methods and use of the compositions herein, sample subdivisions are assayed and the assay results may be mapped or grouped according to the subdivision of their origin, without the use of exogenously added barcode or tag molecules. The resulting datasets have a relatively low proportion of exogenously added information, and do not have subdivision-specifying barcodes or tags, which increases the proportion of sample data in the dataset and decreases the time necessary to generate the sample data.

### CROSS-REFERENCE TO RELATED APPLICATIONS

This document claims priority to US Prov Ser. No. 63/504,730, filed May 27, 2023, which is hereby incorporated by reference in its entirety.

### BACKGROUND

Biomolecule sample analysis has progressed from individual molecule assays to dramatically high-throughput, multi-sample analyses platforms where multiple molecules from multiple subdivisions of a sample or from multiple samples may be analyzed at once.

Though tremendously beneficial, this high degree of throughput presents a challenge, as the individual subdivisions and individual samples need to be distinctly tracked in a given set of reactions. This challenge has been met by adding exogenous markers such as barcodes to identify samples and sample subdivisions.

A drawback of these approaches is that a substantial portion of the assay measurements are occupied by the exogenous labels, such that overall sample measurement efficiency is harmed. Similarly, the number of samples and sample subdivisions to be measured is limited by the availability of distinct or unique labels to specify or distinguish samples or subdivisions. Additionally, barcoding of sample subdivisions such as emulsion droplets or microcapsules requires additional processing steps beyond mere sample barcoding, some of which steps may be incompatible with earlier sample processing, and which are likely to substantially increase processing time and complexity, particularly for processing in emulsion droplets.

### SUMMARY

Disclosed herein are methods of delivering sample subdivisions to discrete positions on an assay region. Some such methods comprise one or more of subdividing a sample into encapsulated subdivision capsules, delivering the encapsulated subdivision capsules to the assay region, and releasing contents of the encapsulated subdivision capsules at the assay region such that contents of the encapsulated subdivision capsules do not intermingle.

Also disclosed herein are methods of sorting sample assay results. Some such methods comprise one or more of comprising subdividing a sample to a first sample set and a second sample set, assaying the first sample set at the first location so as to associate a first location identifier to sequences of the first sample set, assaying the second sample set at the second sample location so as to associate a second location identifier to assay results of the second sample set, associating assay results having the first assay identifier to the first sample set, and associating assay results having the second sequence identifier to the second sample set.

Similarly, disclosed herein are methods of mapping nucleic acids to a single cell. Some such methods comprise one or more of encapsulating a cell into a solid capsule having an aqueous core; subjecting the cell to lysis to yield cell nucleic acids in the aqueous core; subjecting the cell nucleic acids to sequencing library preparation; loading the capsule onto a flow cell; releasing the cell nucleic acids from the capsule; and sequencing the cell nucleic acids.

A location identifier comprises positional or temporal information associated with target information such as target sequence information. That is, upon gathering target sequence information, one also obtains information relating to the location from which the sequence was obtained or the time or order at which the sequence was obtained. Thus rather than or in addition to relying upon a common barcode, one may assign a target read a positional ‘barcode’ such that the position or sequential or temporal order at which a target sequence is obtained may be used much like a barcode to associate that target sequence with a second target sequence or a group of target sequences sharing common or similar positional or temporal information.

Also disclosed herein are approaches for library generation wherein the library generation does not comprise a sample subdivision indicative barcode. Some such approaches similarly do not require other labeling. Through practice of the disclosure herein, target reads are nonetheless assigned to sample subdivisions of origin without sample subdivision indicative barcode addition prior to target determination. Sample barcodes, indicative of sample of origin or sample treatment, for example, are compatible with and employed in at least some of these embodiments, for example so as to allow bulked sequencing of libraries generated from distinct samples such as distinct individuals or distinct tumors, or even from entirely unrelated samples on a single sequencing device. However, often no subdivision specific or indicative barcode, such as an emulsion droplet specific barcode or microcapsule specific barcode, is required or used in some embodiments herein.

Unbarcoded sample subdivisions are in some cases delivered to a surface or channel at random, that is in a spatially or temporally unordered manner. Alternately, in some cases sample subdivisions are delivered in a directed or ordered manner. That is, a position is selected and a particular sample subdivision is delivered to that position, such that target reads arising from that position can be mapped or assigned to a particular sample subdivision. Locations are in some cases identified through imaging, such as concurrent with sample subdivision delivery. Similarly, in some cases sample subdivisions are delivered in an ordered manner, such as to a channel for sequencing, such that the order of emergence of groups of target sequences may be aligned to order of sample subdivision delivery.

Consistent with the methods disclosed herein are datasets comprising date generated through practice of the methods herein, and sample libraries such as nucleic acid libraries consistent with practice of the methods herein.

Also disclosed herein are nucleic acid datasets comprising a plurality of nucleic acid sequence sets, wherein separate nucleic acid sequence sets of the plurality of nucleic acid sequence sets are distinguished by non-nucleic acid positional tags indicative of a common position where nucleic acids of separate nucleic acid sequence sets were sequenced, and wherein the separate nucleic acid sequence sets do not differ in exogenously added set-identifying nucleic acid barcode sequences.

Also disclosed herein are nucleic acid datasets comprising sample nucleic acid sequence of a first nucleic acid sequence set arising from a first subdivision and a second nucleic acid sequence set arising from a second subdivision, wherein the first nucleic acid sequence set comprises nucleic acids associated with a first sequencing location and wherein the second nucleic acid sequence set comprises nucleic acids associated with a second sequencing location, and wherein the first nucleic acid sequence set and the second nucleic acid sequence set are not distinguished by set-identifying exogenously added nucleic acid tags.

The datasets and libraries variously comprise at least 10, at least 50, at least 100, at least at least 500, at least 1,000, at least 5,000, at least 10,000, at least 50,000, at least 100,000, at least 500,000, at least 1,000,000 or more than 1,000,000 molecules such as nucleic acid molecules, or data constituents such as nucleic acid sequences.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows pore usage by DNA fragments derived from a single capsule on a MinION flow cell over time.

FIG. 2 shows a different view of the well of origin of the reads obtained in the first 20,000 seconds of the sequencing run which result is presented in FIG. 1.

### DETAILED DESCRIPTION

### Introduction

Disclosed herein are compositions, methods and systems relating to the containment and delivery of subdivided portions of a sample to an assay site or destination site. The portions are optionally processed prior to delivery, such as through cell lysis, protein isolation or nucleic acid library generation so as to facilitate analysis at a downstream assay site. Through practice of the disclosure herein, the physical proximity of a portion of a sample is preserved through a sample preparation process, such that proximity of the processed sample constituents delivered to an assay site or a destination site is indicative of those sample constituents arising from a common portion of the sample.

Often, the subdivided portion of a sample comprises a single cell of the sample, though other subdivided portions of a sample are also consistent with the disclosure herein. Through practice of the disclosure herein, the subdivided portion of the sample such as a bacterial or eukaryotic cell, or viral particle, cell organelle, or population of cells, viral particles or organelles, or purified fraction of a sample or any other portion of a sample, maintains its physical proximity throughout any sample processing, such that upon delivery to an assay site or other destination site, the physical proximity is preserved such that molecules in proximity at the assay site or other destination site are reliably inferred to have arrived from a common subdivided portion of the sample. Accordingly, some sample processing approaches do not comprise or do not require sample subdivision barcoding prior to delivery to an assay site or other destination site, such as a site onto which a subdivided portion of a sample is released. Samples are in some cases barcoded to add a sample identifying barcode common to, identifying or unique to a particular sample. Such a barcode may be added prior to the encapsulation or sample subdivision generation process, and is distinct from a barcode that would identify or distinguish one sample subdivision from another.

Proximity at the assay site or other destination site may be used as an indicator of common origin in a common subdivided portion of a sample, such as a cell or other portion. Thus, in some cases common origin for molecules of a common subdivided portion of a sample is inferred without subjecting the subdivided portion of the sample to any subdivision-specific or subdivision-indicating marker, such as a stain, label, or tag such as a nucleic acid tag. Instead, positional information at the assay site or other destination site is sufficient as an indicator of common origin in a common subdivided portion of a sample, even when the structural integrity of the subdivided portion of the sample does itself maintain proximity of the portion constituent molecules, as is the case when a cell of a subdivided portion is lysed, as often occurs pursuant to sample processing.

### Encapsulation

A number of encapsulation approaches are consistent with the disclosure herein. In some cases any encapsulation approach sufficient to maintain the proximity of a sample subdivision while allowing its delivery to an assay site or other destination site is consistent with the disclosure herein. Accordingly, in some cases sample subdivisions are stored in wells of a microwell plate or surface, or are stored in aqueous droplets of a water in oil emulsion. Samples are then subjected to processing through addition of reagents to the subdivision, as allowable in light of dilution constraints, delivery constraints associated with delivering reagents to aqueous droplets of a water in oil emulsion. Or manipulation constraints associated with delivery to and then, in some case, removal from static wells for delivery to an assay site or other destination site. Samples are in some cases barcoded to add a sample identifying barcode common to, identifying or unique to a particular sample. Such a barcode may be added prior to the encapsulation or sample subdivision generation process, and does not in most cases distinguish one sample subdivision from another, but may facilitate bulking of multiple samples for sequencing on a common flowcell.

Technology relating to delivery of sample subdivisions to wells or of generation of emulsions from aqueous samples is well established. Microwell technology is discussed, for example, in WO2017/019456, published Feb. 2, 2017 and issued as U.S. Pat. No. 10,913,975 on Feb. 9, 2021, the contents of which are hereby incorporated by reference in their entirety. Similarly, emulsion technology is discussed in a number of references, for example WO2004/04038363, published May 6, 2004, and issued, for example, as U.S. Pat. No. 7,129,091 on Oct. 31, 2006, which is hereby incorporated by reference in its entirety.

Alternately, encapsulation is effected through enclosing a sample subdivision portion in the interior of a microcapsule, such as a degradable, porous hydrogel microcapsule shell having an aqueous interior. Such an encapsulation approach allows replacement of the microcapsule interior chemical environment through incubation of the microcapsule in a carrier liquid having the desired characteristics, such that the carrier may equilibrate with the microcapsule via diffusion across the porous hydrogel. Multiple condition replacements may be effected, such as introduction of lysis conditions, reverse transcription conditions, library packaging or other reaction conditions. These replacements do not require serial dilution of the sample subdivision volume or droplet merger. Also, delivery of the processed sample subdivision does not require either individual manipulation of microwell contents, or the contacting of an assay site or other destination site to a hydrophobic carrier pursuant to emulsion delivery. Instead, porous microcapsules harboring processed sample subdivisions are delivered in an aqueous carrier to an assay site or other destination site, and are then degraded at the assay site or other destination site, such that upon release of microcapsule contents, proximity of the released molecules, on the assay site or other destination site may serve as an indicator of a common microcapsule of origin.

Encapsulation technology is discussed, for example, in WO2020/255108, published Dec. 24, 2020, and also published in the US as 2020/0400538 on Dec. 24, 2020, the contents of which are hereby incorporated by reference in their entirety, as well as US2021/0268465, published Sep. 2, 2021, and U.S. Pat. No. 8,765,485, published Jul. 1, 2014, both of which are hereby incorporated by reference in their respective entireties. Common features of some capsulation technologies are that samples are subdivided into aqueous cores of microcapsules, such as porous hydrogel microcapsules, that are readily degraded to release their contents, such as at an assay site or other destination site.

### Processing

A broad range of sample subdivision processing reactions are consistent with the compositions, methods and systems herein. In particular, the disclosure is tolerant to processing steps that disrupt proximity maintaining features of a sample subdivision, such as a cell membrane or cell wall, a nuclear membrane, an organellar outer or inner membrane or thylakoid, a viral capsid or particle exterior, or other component that maintains structural integrity of a sample constituent. Despite such a disruption, the physical proximity of the sample subdivision contents is maintained in the microcapsule, such that upon delivery to an assay site or other destination site, the physical or temporal, or both physical and temporal proximity of the sample subset is preserved.

Processing may be effected by addition of reagent volumes to a sample subdivision so as to create reaction conditions in the sample subdivision conducive to the desired processing reaction. This involves successively diluting the sample subdivision such that conditions within the subdivision are conducive to one or more subsequent processing steps. For example, a cell captured in a sample subdivision may be lysed by adding to the sample subdivision a reagent bolus sufficient to create lysis conditions in the combined sample subdivision volume. The process may be repeated by adding a second bolus sufficient to create, for example, reverse transcription conditions and deliver a reverse transcriptase enzyme. Successive processing steps such as packaging nucleic acid templates for sequencing may comprise delivery of additional reagents, dilution to change reaction conditions, or both.

Reagent delivery is effected through direct addition of reagent volumes, either by injection to microwells, or through droplet merger, injection, or flow merger and droplet recovery for emulsions such as water in oil emulsions.

A consequence of this reagent volume addition approach is that the sample subdivision increases, in some cases substantially, pursuant to each reagent delivery step. This increase in volume may in some cases render the sample subdivision cumbersome to manipulate, and may increase the area occupied by the sample subdivision when the sample subdivision is deposited onto an assay site or other destination site.

Alternately, reagent delivery is effected through reagent volume replacement. Through this approach, sample subdivisions are sequestered in microcapsules or other containers that allow reagent volume exchange, such as via pores in a porous microcapsule shell. After sample subdivision in such microcapsules or other containers that allow reagent volume exchange, the microcapsules or other containers are washed in an excess of a reagent volume. As the microcapsules or other containers are porous to small molecules but not to nucleic acids of, for example, 500 bp or larger or to comparably sized macromolecules (alternately 10, 20, 30, 40, 50, 100, 200, 300, 400, 500, 750, 100 or greater than 1000 bp) reagent buffers can readily diffuse through the pores. Accordingly, by placing the microcapsules or other porous containers in an excess of a target reagent volume, the sample subdivision volumes will equilibrate to the target reagent conditions. This effective reagent volume replacement is accomplished without iterative increases in sample subdivision volume, and can be performed a number of times limited by microcapsule integrity rather than by any negative effects of dilutive volume increase.

Consequently, a broad range of processing reactions, to be optionally performed in distinct successive steps, are facilitated by the practice of the approaches herein.

A partial list of processing steps consistent with the disclosure herein comprises cell or membrane lysis, cell wall degradation, partial or complete cell membrane permeabilization, nuclear or cytoplasmic organellar membrane degradation, protease treatment, RNA degradation, reverse transcription, amplification, end-capping or other library preparation, selective degradation, fluorophore or radiolabel introduction, in vitro transcription, in vitro translation, endonuclease treatment, exonuclease treatment, cross-linking, ligation, or other molecular biological or biochemical manipulations.

Barcoding is also consistent with the processing steps facilitated by the approaches herein. However, as the physical proximity of the reagent is maintained throughout the processing steps, delivered reagents may be identified as arising from a common sample subdivision through their physical proximity, temporal proximity, or both physical and temporal proximity of delivery onto an assay site or other destination site. In some cases, barcoding is used in combination with proximity information to identify sequence reads as arising from a common microcapsules.

### Delivery to an Assay Region

Subsequent to encapsulation of a sample subdivision, and in many cases one or more processing steps, the processed sample subdivision is delivered to an assay site or other destination site. Molecules in a microcapsule may be subjected to a broad range of processing steps, ranging from cell lyses and purification from cell contents that are degraded and may diffuse from the microcapsule, to nucleic acid library preparation. Processing may comprise reverse transcription or treatment to generate DNA strands of a size suitable for bridge amplification. Processing may comprise targeted or untargeted nucleic acid amplification. Library preparation may comprise adapter addition, such as P5 and P7 adapter addition consistent with bridge amplification colony or cluster formation, nanopore library preparation, circularization pursuant to rolling circle cluster formation or smartbell long read library preparation. In some cases all nucleic acids are targeted for library generation. Alternately, in some cases only a subset are targeted, for example so as to facilitate selective measurements.

A number of delivery approaches are consistent with the disclosure herein. Sample subdivisions contained in microwells wells of a microplate may be delivered to an assay site or other destination site by micro-pipetting or injection or by direct transfer via wicking or vacuum transfer or spin-coating or through, for example, holes in well bottoms. Delivery can be driven by, for example, gravity, centrifugal force, application of pressure, capillary force or electrostatic force.

Sample subdivisions contained in emulsions such as water in oil emulsions may be flowed directly to an assay site or other destination site, where the aqueous droplets are allowed to settle on the assay site or other destination site while the hydrophobic carrier is drawn off. Alternately, emulsion droplets are deposited by being encased in hydrogel chambers pursuant with deposition on a surface. In some cases the emulsion is broken using a reagent such as a de-emulsifier. A challenge to this approach is to prevent mixing of aqueous droplets in the sample subdivision deposition process. In some cases deposition is performed in a carrier having a viscosity greater than that of water, so as to reduce capsule contents diffusion prior to deposition.

Microcapsule sample subdivisions may be manipulated much like emulsions in that they readily flow in bulk. An advantage of microcapsules, particularly in an aqueous carrier, is that they may be delivered to an assay site or other destination site in bulk much like emulsions, but they do not require the careful removal of the hydrophobic carrier, which may be incompatible with downstream analyses. Furthermore, microcapsules may be fixed to a surface. The challenge of microcapsules, that they encase sample subdivisions in a solid shell, is readily overcome to facilitate deposition. In some cases lysis is performed in a carrier having a viscosity greater than that of water, so as to reduce capsule contents diffusion prior to deposition. Exemplary microcapsules comprise a biodegradable shell such as a shell susceptible to enzymatic degradation, such that their sample subdivision contents are readily released under biological conditions. Alternately, microcapsules may be degraded using heat, shearing, sonication, chemical hydrolysis or other approaches.

Exemplary core-shell compositions comprise methacryloyl butyryl or acryloyl butyryl or similarly modified dextran polymer scaffolds. Such compositions, and many similar compositions comprising a carbohydrate monomer, are readily degraded using dextranase or other glycolytic enzyme. Dextran polymers of 10 kDa to 2 MDa are consistent with the disclosure herein, as are polymers outside of these ranges.

A process for manufacturing a hydrogel composition including a plurality of microcapsules, involves one or more of the following steps, using dextran as an example: (a) emulsifying in a droplet generation device (i) a first aqueous solution comprising a first polymer, and (ii) a second aqueous solution comprising a second polymer, in an oil, wherein: the first polymer comprises dextran modified with (i) conjugated methacryloyl cross-linking moieties and (ii) conjugated butyryl moieties; the second polymer comprises dextran not modified with conjugated methacryloyl cross-linking moieties and not modified with conjugated butyryl moieties; the first aqueous solution and/or the second aqueous solution comprises a biological entity; monodisperse water-in-oil droplets containing the first polymer, the second polymer and the biological entity are generated; and an aqueous two-phase system is formed inside the water-in-oil droplets in which a liquid core is completely surrounded by a liquid shell and the biological species is preferentially distributed in the liquid core; and (b) exposing the microcapsules to cross-linking conditions that conjugate at least a portion of the methacryloyl moieties in the first polymer, thereby forming a hydrogel shell surrounding a core in a plurality of microcapsules.

A broad range of hexose or pentose monomer units, such as dextran and cellulose, are consistent with the hydrogel compositions herein. Charge neutral non-ionic polysaccharides are preferred in some embodiments, as are polysaccharide units bound by glycosidic bonds. Glycan-forming monosaccharide units are often preferred, though alternatives are also contemplated. An exemplary polysaccharide category is the of polysaccharides collectively referred to as ficoll.

A cross-linking moiety or moieties used to facilitate formation of the hydrogel shell independently are chosen from an acryloyl group or a substituted acryloyl group, such as acryloyl, or methacryloyl, or acryloyl and methacryloyl.

A common feature of many of the sample subdivision deposition approaches is that at least some sample subdivision volumes are not intermingled pursuant to the deposition process, such that physical, temporal, or physical and temporal information about adjacent sample subdivision products may be used to infer their common origin in a common sample subdivision. This sample subdivision preservation is in some cases absolute, such that sample subdivisions are not intermingled.

Alternately, the sample subdivision preservation is in some cases partial, such that sample subdivisions sharing what are likely to be common or similar contents may intermingle, while sample subdivisions harboring distinct contents are prevented from having their volumes intermingle pursuant to deposition. For example, cells sharing a common cell surface identifier may in some cases be harbored in sample subdivision volumes that intermingle pursuant to deposition, while they are held distinct from sample subdivisions harboring cells that do not posses that surface identifier. In some cases lysis is performed under conditions so as to reduce capsule contents diffusion prior to deposition in a carrier having a viscosity greater than that of water.

In either case, a result of deposition is that proximity of analytes subjected to analysis is informative of the relative identity of their origin within a sample. That is, temporally, spatially, or temporally and spatially adjacent analytes are often inferred to arise from a common sample subdivision or subdivisions harboring common or similar sample material. Conversely, temporally, spatially, or temporally and spatially distant analytes are inferred to arise from distinct sample subdivisions.

In some case, a first sample set or sample subdivision is deposited at a first position or assayed at a first time period, and the assay results are associated with the first position or first time period information. A second sample set or sample subdivision is deposited at a second position or assayed at a second time period, and the assay results are associated with the second position or second time period information. Data arising from the first position or the first time period is attributed to the first sample set or sample subdivision, while data arising from the second position or the second time period is attributed to the second sample set or sample subdivision. The first position and the second position are in some cases concurrently assayed.

Alternatively or in combination, in some cases a first sample subdivision is deposited at a first position and a second sample subdivision is deposited at a second position. Density of target molecules arising from the first sample subdivision may decrease, for example radially from a first sample deposition point, while target molecules arising from the second sample subdivision may decrease, for example radially from a second sample subdivision deposition point. Individual target molecules are assigned to a sample subdivision group according to the local density of their vicinity or by the nearest local density peak. That is, a target molecule or sequence of a target molecule arising from a region of locally high molecule or read density is assigned to a common origin or a common sample subdivision as other target molecules or sequences of a target molecules of the locally high-density region. Similarly, in some cases a target molecule or sequence of a target molecule arising from a region of low density is assigned to a common origin or a common sample subdivision as other target molecules or sequences of a target molecules in the nearest locally high-density region. In alternative cases a target molecule or sequence of a target molecule arising from a region of low density is discarded from analysis or unassigned. Determination of ‘high density’ or ‘high density region’ borders are in some cases accomplished using predetermined density thresholds, predetermined distances from a center of high density, or predetermined fall-offs in density. As an alternative to using predetermined values, one may determine values for a given array of target molecules based upon determinations made a posteriori upon evaluation of target or sequence read distributions for a given set of sample subdivisions on a surface or temporally. In various embodiments, the radius defining a high-density region is no more than, at least, about or exactly 1 um, 2 um, 5 um, 10 um, 20 um 50 um, 100 um, 200 um, 500 um, 750 um, 1000 um, 1500 um, 2000 um, 5000 um or greater. Some high density regions range in size from, for example, 1 um to 2000 um, 5 um to 1000 um, or 50 um to 500 um. In some embodiments the radius defining a high-density region approximates the radius of the sample subdivisions from which the high-density regions are derived. Some high-density regions are no more than 10%, 20%, 30%, 40%, 50%, 75% or 100% greater than the sample subdivisions from which the high-density regions are derived. Some high-density regions are no more than 10%, 20%, 30%, 40%, or 50%, smaller than the sample subdivisions from which the high-density regions are derived. In various embodiments a high-density region is identified as reaching its edge when the local target read density is no more than any of, for example 99%, 95%, 90%, 85%, 80%, 75%, 70%, 65%, 60%, 50%, 45%, 40%, 35%, 30%, 25%, 20%, 15%, 10%, 5% or less than 5% of a central density or a local maximum density. In some cases determinations are added by assaying location of a dye or fluorophore co-partitioned with a sample subset, particularly one that differs from a second dye or fluorophore co-partitioned with a second sample subset, such that the distribution of the two differing dyes may serve as a marker for the distribution of target reads corresponding to their respective sample subsets. Determinations are made alone or in combination with other information, such as sequence identity information, sequence GC content, read length, organism of origin, as may be informative in heterogeneous sample such as samples comprising pathogens or arising from heterogeneous sources such as environmental samples or gut microflora, or in some cases sample tags or barcodes.

Some systems herein effect sample subdivision sorting on surfaces exhibiting an occupancy of no greater than, no less than, about or exactly 10% 20%, 30%, 40%, 50%, 60%, 70%, 80% 90% or greater than 90%.

Sample subdivision sorting is in some cases effected through surface partitioning. Some surfaces are partitioned into wells or planar partitions configured to accommodate no more than a single sample subdivision per partition. In some such systems, sample subdivision partitioning is effected even under high degrees of partition occupancy, for example at least 50%, 60%, 70%, 75%, 80%, 85%, 90%, 95%, or greater than 95%.

Similarly, sample subdivision sorting is in some cases effected through temporal partitioning, such as that effected by delivering sample subdivisions to an assay region such as an assay channel in a linear sequence, with a first subdivision and a second subdivision separated by a region of locally low-density target occupancy or lacking targets. In some such systems, sample subdivision partitioning is effected by separating sample subdivisions by empty carrier of a volume of, for example at least 10%, 20% 30%, 40%, 50%, 60%, 70%, 75%, 80%, 85%, 90%, 95%, or greater than 95% of that of the sample subdivisions.

A benefit of some of these sorting approaches is that samples may be sorted into target molecules arising from a first sample subdivision and a second subdivision without requiring unambiguous barcoding of individual target molecules within a subdivision, with only partial barcoding or barcoding of only a subset of targets in a sample subdivision, or in some cases without any barcoding prior to sample deposition. Similarly, samples are in some cases sorted without any additional tags such as sample subdivision fluorophore tags or other indicators of sample subdivision volume or contents distribution on a surface or temporally in a channel.

### Assay Regions

A number of assay regions or destination sites are compatible with the disclosure herein. The diversity of assay sites or destination sites compatible with the disclosure herein reflects the diversity of analyses and sample analytes to be assayed through various embodiments of the disclosure herein. Assay sites contemplated herein may be compatible with one or more of nucleic acid analysis, protein or other polypeptide analysis, carbohydrate analysis, bioenergetic analysis pursuant to mitochondrial electron transport or chloroplast photosynthetic activity, lipid analysis, molecular signaling analysis such as kinase activity or phosphorylation status, cell surface marker or other molecular analysis.

For the case of nucleic acid analysis in samples, exemplary assay regions comprise flow cell surface positions, or channel openings for nanopore analysis. Sample subdivisions may be distributed at spatially distinct positions on a flow cell surface, and then their contents released and bound to the surface such that adjacent molecules on a surface are likely to have arisen from a common sample subdivision. Flow cell surfaces may be unstructured, or alternately may have patterned binding positions or even wells.

In exemplary embodiments of structured flow cells, the patterned binding positions or wells are large enough to accommodate the sample subdivisions, such as at a proportion of one sample subdivision per patterned binding position or well. If dilution is used to effect successive processing reactions, the successive dilutions are selected to keep the final sample subdivisions from being larger in cross-sectional area than the patterned binding positions or wells to which their contents are to be bound. In these cases target sequence arising from a common pattern feature are assigned to a common sample subdivision. Alternately, in some cases patterned binding positions or wells may accommodate multiple sample subdivisions.

Structured flow cells may comprise ‘fenced’ patterning, such that regions configured to accommodate a microcapsule are bordered by elevated walls or enclosed in recessed depressions on the flow cell. These walls may in some cases facilitate microcapsule retention at a release site. Similarly, flowcell walls may serve to interrupt diffusion of microcapsule released contents, such that microcapsule contents are retained at a site of microcapsule degradation rather than diffusing to locations elsewhere on the flowcell. Such a configuration may restrict diffusion of microcapsule contents to no more than 30%, no more than 20%, no more than 10%, no more than 5% or less of the total microcapsule payload. Similarly, such a configuration may restrict diffusion of microcapsule contents to contents of no greater than 5 kb, no greater than 4 kb, no greater than 3 kb, no greater than 2 kb, no greater than 1 kb, no greater than 500 bp, no greater than 250 bp, no greater than 100 bp, or no greater than 50 bp.

Flowcells having ‘fencing’ may also show smaller scale patterning, such as patterning to produce single colony sized binding sites. This smaller scale patterning may be at the level of nucleic acid adhesion only—that is, of binding sites separated by borders that do not bind nucleic acids. Alternately, patterning may comprise microwells having walls, such as to contain individual colonies. Generally, if such wells exist, their walls are substantially smaller than the ‘fencing’ walls produced to contain microcapsules prior to degradation, or to also preclude diffusion of microcapsule contents subsequent to lysis. Exemplary flowcell fencing is configured to harbor roughly spherical microcapsules of a diameter of at least, at most or about 10, 20, 30, 40, 50, 75, 100, 125, 150, 200 or greater than 200 um.

Unstructured flow cells are often loaded with sample subdivisions or microcapsules such that the sample subdivisions are not tightly packed. In these cases there is substantial space between adjacent sample subdivisions. The space between sample subdivisions is reflected in the footprint their contents leave on the flow cell, such that distance between groups of bound molecules may facilitate sorting these molecules into sample subdivisions of origin. Alternately, a few, some, a majority or all of the sample subdivisions may abut at least one other sample subdivision, such that there is no flow cell gap between some or all of the sample subdivision footprints on the flow cell. In these cases, one or more approaches may be used to assign border molecules to a sample subset of origin, or to exclude them from analysis. Some such approaches may comprise identification of a microcapsule or subdivision content spot ‘center’ position and including only sequence reads that are located within a radius of the center position, or including reads beyond such a radius if they abut an empty edge of a spot of colonies. In various embodiments, the radius is no more than, at least, about or exactly 1 um, 2 um, 5 um, 10 um, 20 um 50 um, 100 um, 200 um, 500 um, 750 um, 1000 um, 1500 um, 2000 um or greater. In various embodiments a high density region is identified as reaching its edge not at a set radius but when the local target read density is no more than any of, for example 99%, 95%, 90%, 85%, 80%, 75%, 70%, 65%, 60%, 50%, 45%, 40%, 35%, 30%, 25%, 20%, 15%, 10%, 5% or less than 5% of a central density or a local maximum density. In some cases a local density drop-off, such as one above, is used to define a radius. Colonies or reads that fall at a junction of adjacent microcapsule or subdivision spots may be excluded from further analysis, or subjected to more stringent analysis such as assessment of their sequence contents.

Nucleic acid analyses using channel openings or nanopore analyses are also compatible with the disclosure herein. In these cases, sample subdivisions are delivered either temporally to a single channel opening or nanopore, or spatially to a plurality of channel openings or nanopores, or both temporally and spatially to a plurality of channel openings or nanopores.

Sample subdivisions are delivered in some cases such that there is a temporal separation between analysis of contents. The temporal separation in data output may demarcate borders between contents of distinct sample subdivisions. That is, molecules measured in temporal proximity to one another are assigned to a common sample subdivision of origin, while molecules whose measurement is separated by a period during which no measurements are made are assigned to distinct sample subdivisions. This temporal separation may be used in alternative to or in combination with spatial separation to assign data for individual molecules to common or distinct sample subdivisions.

The above-mentioned flow cell and nanochannel discussions are made in the context of nucleic acid synthesis but are broadly applicable to analytes that are detected on a spatial plane or on a temporally linear channel. For example, samples may be analyzed by iterative preparation followed by delivery in series of sample subdivisions to a nanochannel, or an array of nanochannels, that feeds into a mass spectrometric analysis of proteins or peptide fragments, or of lipids or starches, or any combination of these alone or in combination with additional reagents.

For both temporal and spatial separation of sample subdivisions, smaller sample subdivisions facilitate higher throughput. Accordingly, in exemplary embodiments reagent replacement is used to enable iterative processing steps without concomitant increase in sample subdivision volume.

### Exogenous Barcode-Free Nucleic Acid Libraries

Some nucleic acid libraries generated through practice of the disclosure herein are characterized in that individual members do not share subdivision-identifying tags or markers, but that sequencing arising from these libraries may nonetheless be accurately assigned to subdivisions of origin. This is accomplished because the nucleic acid library constituents are generated and configured so as to maintain positional information throughout the sample processing and library generation and continuing through library sequencing. The positional information (temporal, spatial, or both temporal and spatial) is preserved and assigned to the sequence data arising from the library, such that sequence reads assigned the same or similar positional information can confidently be attributed to the same sample subdivision despite not sharing a sample subdivision-identifying barcode or tag.

Consequently, nucleic acid library constituents are not encumbered by the requirement of harboring unique or sample subdivision identifying barcodes, or the requirement that their synthesis comprise a step of barcode addition. Depending upon library constituent length and oligo size, the proportion of the library dedicated to oligo tags may occupy as much as 15% or more than 15% of a total library constituent length, such as at least or more than 1%, 2%, 5%, 10%, 15%, 20%, 30%, 40%, or of total library length. Accordingly, the disclosure here enables increases in the proportion of biologically relevant nucleic acids in a nucleic acid library, and decreases the proportion of exogenously added nucleic acids generated pursuant library generation. Similarly, the disclosure herein enables decreases in library preparation time and complexity by not requiring barcode addition. In some cases, the library comprises no sample subdivision indicative oligo, or no more than 1%, no more than 2%, no more than 3%, no more than 4%, no more than 5%, no more than 6%, no more than 7%, no more than 8%, no more 9%, or no more than 10% of a sample library. These efficiencies are accomplished while nonetheless assigning a substantial proportion of library molecules or other molecular output to a sample subdivision of origin, such as a cell or there source herein. The proportion is in some cases at least 50%, at least 60%, at least 70%, at least 80%, at least 90%, at least 95%, at least 99%, or up to 100% of the sequence reads or other molecular output signals assigned to a sample subdivision of origin.

The datasets and libraries contemplated herein variously comprise at least 100, 500, 1,000, 5,000, 10,000, 50,000, 100,000, 500,000, 1,000,000 or more than 1,000,000 molecules such as nucleic acid molecules, or data constituents such as nucleic acid sequences.

### Exogenous Barcode-Free Capsule Specific Data

Some datasets generated through practice of the disclosure herein share the feature that their individual molecular data are confidently assigned to a sample subdivision of origin without the use of an exogenously added sample subdivision marker. That is, sample constituents are allotted to sample subdivisions, lysed, processed, and their nucleic acids are sequenced and for each sample subdivision are assigned to a cell of origin without the need for a sample subdivision identifying barcode or other exogenously added tag.

A number of benefits arise from these barcode-free approaches. In particular, sample processing can be performed without a step comprising adding a sample subdivision identifying or unique barcode to the subdivision or annealing it to nucleic acids of the sample subdivision or otherwise incorporating it into the nucleic acids of the subdivision. Thus the processing pipeline is streamlined, in terms of the number of steps to be performed, the cost, and the time necessary to complete sample processing.

Furthermore, the number of sample subdivisions to be assayed is not limited by the number of available sample subdivision tags such as barcodes. There are, for example, 4e6 or 4096 unique hexamer tags, and 4e8 or 65,536 octamer tags. If one is to rely exclusively upon hexamer or octamer tags to identify a sample subdivision of origin, then for a given run one may distinguish only a number of sample subdivisions governed by the diversity of the marker population.

Admittedly, longer oligo markers may be used, but at a nontrivial cost in terms of generation of the oligos, delivery of the oligos to sample subdivisions, and proportion of overall nucleic acid read length occupied by oligo sequence. Furthermore, the assignment of nucleic acids to sample subdivisions is vulnerable to errors in sequencing the oligomers.

Through use of the disclosure herein, in contrast, the number of sample subdivisions to which molecular data is to be sorted is limited only by the available flow cell area or the time to be spend sequentially feeding sample subdivisions into a nanochannel or array of nanochannels. Accordingly, much larger numbers of sample subdivisions may be assayed, and much more rare sample subdivision phenomena may be detected.

Similarly, as alluded to above, sample data collection is not encumbered by the requirement of reading or identifying barcodes in sample molecular output data such as sequence reads. Depending upon read length and oligo size, the proportion of read length dedicated to oligo tags may occupy as much as 15% or more than 15% of a total read through conventional methods. Accordingly, the disclosure here enables increases in the proportion of biologically relevant sequence in a nucleic acid output, and decreases the proportion of exogenously added sequence generated pursuant to a sample run. In some cases, the sample run output comprises no sample subdivision indicative oligo sequence, or no more than 1%, no more than 2%, no more than 3%, no more than 4%, no more than 5%, no more than 6%, no more than 7%, no more than 8%, no more 9%, or no more than 10% of a sample sequence output. These efficiencies are accomplished while nonetheless assigning a substantial proportion of sequence reads or other molecular output to a sample subdivision of origin, such as a cell or there source herein. The proportion is in some cases at least 50%, at least 60%, at least 70%, at least 80%, at least 90%, at least 95%, at least 99%, or up to 100% of the sequence reads or other molecular output signals assigned to a sample subdivision of origin.

The datasets and libraries contemplated herein variously comprise at least 100, 500, 1,000, 5,000, 10,000, 50,000, 100,000, 500,000, 1,000,000 or more than 1,000,000 molecules such as nucleic acid molecules, or data constituents such as nucleic acid sequences.

### Uses of Cell-Specific Information

High throughput cell or sample subdivision-specific data has a number of valuable uses for health and scientific discovery. Tumor samples, for example, exhibit dramatic cell to cell variation in their accumulated mutations, transcriptome expression and cell surface proteins.

Individually assaying tumor and adjacent healthy cells as to their transcriptome, nuclear and mitochondrial genomes, provides valuable insight into which mutations occur in combination and which should be targeted for therapeutic treatment.

Diagnosis, particularly of disease resulting from emerging or rapidly evolving pathogens, is also facilitated by high throughput, rapid, efficient sample subdivision analysis. By separating individual cells and viral particles, one may rapidly identify and sequence unknown pathogens such as viral pathogens, or quickly determine the antibiotic resistance status of individual members of a bacterial pathogen population in an individual. One may also concurrently determine, through transcriptome analysis, the transcriptional response of infected cells to pathogen attack.

### Methods

Consistent with the above, disclosed herein are methods, compositions, systems and datasets related to sample subdivisions and their analysis.

Some methods consistent with the disclosure herein relate to delivering sample subdivisions to discrete positions on an assay region. Some such methods comprise one or more of comprising subdividing a sample into encapsulated subdivision capsules, delivering the encapsulated subdivision capsules to the assay region, and releasing contents of the encapsulated subdivision capsules at the assay region such that contents of the encapsulated subdivision capsules do not intermingle, or such that no more than 1%, 2%, 5%, 10%, 20%, 30%, 40% or 50% of the contents of the capsules are intermingled, or that contents do not intermingle substantially until after 1, 2, 5, 10, 20, 50, 100, 200, 500, 1,000, 2,000, 5,000, 10,000, 20,000, 30,000, 40,000, 50,000, 60,000, 70,000, 80,000, 90,000, 100,000 or more than 100,000 seconds.

Samples variously comprise nucleic acids, cells, polypeptides, lipids, carbohydrates or other constituents disclosed herein or consistent with biological samples. Assay regions to which samples or sample subdivisions are delivered variously comprise flow cell sites, a microchannel or a plurality of microchannels, or a cell culture medium sch as a culture plate.

Releasing contents of the encapsulated subdivisions distinctly variously comprises releasing contents of the encapsulated subdivision capsules at distinct locations of the assay region, or at temporally distinct times, or at both distinct locations and temporally distinct times. Releasing is effected through, for example, biodegrading the encapsulated subdivisions via, for example, enzymatic treatment, or alternately heating, shearing, mechanically degrading or dissolving the encapsulated subdivisions.

The methods variously comprise assaying polypeptides of a first sample subdivision and a second sample subdivision of the encapsulated subdivisions, correlating assay results of the first sample subdivision to a first sample subdivision position and correlating sequences of the second sample subdivision to a second sample subdivision position. Often, this is effected by assigning molecules measured in proximity to a common subdivision of origin, while molecules measured distally to one another are assigned to distinct subdivisions of origin. A first molecule and a second molecule may be categorized as in proximity in some cases if they are adjacent or if they are separated by other molecules, in some cases at a density comparable to the local density of the first molecule or of the second molecule. Similarly, if a first molecule and a second molecule are separated by a region of lower molecule density, in some cases an absence of intervening molecules, the first molecule and the second molecule may in some cases be scored as arising from distinct subdivisions. Alternately or in combination, when subdivisions abut prior to release of their contents their contacted borders may result in a higher density of molecules at their junction. Accordingly, in some cases a first molecule and a second molecule are categorized as arising from distinct subdivisions of they are separated by a region of higher molecular density than their local environment.

Separation is variously, spatial, temporal, or both temporal and spatial. That is, in some cases subdivisions are deposited on spatially distinct regions of an assay surface such as a flow cell, or even in spatially distinct wells. Alternately, in some cases subdivisions are deposited in temporal series at a single nanochannel pore, such that temporal gaps in molecule measurement indicate borders between sample subdivision contents. Similarly, in some cases deposition is both temporally and spatially distinct. Accordingly, assay positions variously comprise wells, nanopores or nanochannel openings, patterned or un-patterned flow cell surface locations, or other locations consistent with molecular analysis.

In some cases no measures are taken to limit microcapsule content diffusion. Alternately, efforts to limit diffusion may be taken. In some cases deposition is performed in a carrier having a viscosity greater than that of water, so as to reduce capsule contents diffusion prior to deposition. Alternately or in combination, deposition may be performed as a carrier is evaporated off of a surface or drawn through the surface, so as to minimize or reduce content diffusion or dispersal.

Wells or fenced in regions of a flow cell may be overlain with mineral oil or other liquid that inhibits or precludes diffusion of microcapsule nucleic acids from an initial well or fenced in region to the remainder of the flow cell. A lid or sealant may be applied to seal sample subdivisions into particular wells, depressions or fenced in regions. Regions.

Alternately or in combination, diffusion may be limited temporally, by restricting analysis to only the early arising reads. This approach does not limit diffusion but limits the dataset to sequence reads obtained prior to diffusion having a substantial effect on read data. In some cases reads are only analyzed if they arise within the first 100, 200, 500, 1,000, 2,000, 5,000, 10,000, 20,000, or 50,000 seconds, for example. Alternately or in combination, one may selectively analyze only the first 1%, 2%, 3%, 4%, 5%, 10%, 20%, 30%, 40%, 50%, or 75%, for example, of the reads.

Molecule measurement in some cases comprises sequencing, such as nucleic acid sequencing of nucleic acids of a first sample subdivision and a second sample subdivision of the encapsulated sample subdivisions, and correlating sequences of the first sample subdivision to a first sample subdivision position while correlating sequences of the second sample subdivision to a second sample subdivision position.

A number of sequencing approaches are consistent with the disclosure herein, such as nanopore sequencing, sequencing comprises sequencing by extension, for example sequencing by synthesis or sequencing by binding, rolling circle sequencing, sequencing by ligation or other sequencing approaches.

Alternately or in combination, molecular measurement may comprise protein detection or sequencing, lipid detection, kinase or other signaling activity measurements, energy potential measurements, carbohydrate measurements or other molecular assays.

Molecular measurements from a first position are assigned to a first encapsulated sample subdivision, and measurements from a second position are assigned to a second sample subdivision, such that sequences are assigned to encapsulated sample subdivisions without relying upon exogenously added tags.

As discussed above, a number of processing steps are consistent with the methods herein. Exemplary reactions include cell lysis, nucleic acid amplification, reverse transcription, radiolabeled ATP exposure, such as is used in kinase activity assays, library packaging, or other assays relevant to measurement of any number of molecules or biochemical status states.

Notably, in some cases the reaction does not comprise capsule-specifying sample barcoding. Similarly, in some cases the capsules or sample subdivisions do not comprise capsule-specifying barcodes.

A number of capsule conformations are consistent with the disclosure herein. In some cases the capsules comprise a porous shell and an aqueous core, and the sample subdivisions reside in the aqueous core. The solid shell often comprises a hydrogel.

Sample subdivisions, particularly after processing, may be released through any of a number of approaches, such as degrading the capsules under physiological conditions, such as a pH within a range from 5-9, a temperature within a range from 4-50 degrees Celsius, alone or in combination with enzymatic digestion.

Alternately, as discussed previously, capsules may comprise droplets emulsions such as water in oil emulsions, or may comprise subdivision into wells.

A broad range of sample constituents are consistent with the disclosure herein, such as cells, organelles, viral particles, extract fractions, or any other constituent disclosed herein. Sample subsets variously comprise a single cell or other constituent, or a plurality of cells or other constituents, or in some cases a mixture of constituents, generally drawn from a common source.

Also disclosed are sample libraries such as nucleic acid libraries and molecule measurement datasets such as nucleic acid datasets produced through practice of any of the above-mentioned methods.

Also disclosed herein are methods of sorting assay results so as to reflect sample subdivision of origin. Some such methods comprise one or more of subdividing a sample to a first sample set and a second sample set, assaying the first sample set at the first location so as to associate a first location identifier to sequences of the first sample set, assaying the second sample set at the second sample location so as to associate a second location identifier to assay results of the second sample set, associating assay results having the first assay identifier to the first sample set, and associating assay results having the second sequence identifier to the second sample set. A number of sample constituent molecule assays are consistent with the disclosure herein. In some cases, sample assay results comprise nucleic acid sequence, and the sample comprises nucleic acids. Alternately or in combination, sample assay results comprise protein information, lipid information, kinase activity or other signaling information, metabolic state information, cell surface marker information or other information or molecule assays as disclosed herein.

The first location and the second location are spatially separated, temporally separated, or both spatially separated and temporally separated.

When no exogenous subdivision markers are added, the first location identifier and the second location identifier do not differ by sequence derived from exogenously added sample set identifying nucleic acid oligos.

The first location and the second location are in some cases on a common flow cell. Sequencing or otherwise assaying the first sample set and sequencing the second sample set occur concurrently, such as part of a common sequencing run, for example pursuant to nanopore sequencing or long read rolling circle sequencing as practiced by Pacific Biosciences sequencing. Alternately, the sequencing comprises sequencing by extension, such as sequencing by synthesis or sequencing by binding, or other sequencing approach. In some cases the distinct locations comprise wells.

Subdividing a sample comprises encapsulating the first sample set in a first capsule and the second sample set in a second capsule. Often, the first capsule comprises an outer shell, such as a porous outer shell, and an aqueous core, and wherein the first sample set resides in the aqueous core.

Subdividing in some cases comprises delivering the first capsule to the first location, and releasing the first sample set from the first capsule.

Releasing the first sample set can be effected through any number of approaches disclosed herein or known in the art, such as degrading the outer shell under physiological conditions, for example a pH within a range from 5-9, such as 5, 5.5, 6, 6.5, 7, 7.5, 8, 8.5, or 9.

Similarly, releasing may occur at a temperature within a range from 4-70 degrees Celsius, such as 4, 5, 10, 15, 20, 25, 30, 35, 37, 40, 42, 45, 50, 55, 60, 62, 65, or 70 degrees Celsius. Releasing in some cases comprises enzymatic digestion.

A number of subdivision contents are consistent with the disclosure herein. In some cases a first sample set comprises a first cell, and the second sample set comprises a second cell.

Also disclosed are sample libraries such as nucleic acid libraries and molecule measurement datasets such as nucleic acid datasets produced through practice of any of the above-mentioned methods.

Also disclosed herein are methods of mapping molecules such as nucleic acids to a single cell. Some such methods comprise one or more of encapsulating a cell into a subdivision such as a solid capsule having an aqueous core, a well or a emulsion droplet; subjecting the cell to lysis to yield cellular contents such as nucleic acids in the aqueous core; subjecting the cellular contents such as nucleic acids to assay preparation processing such as sequencing library preparation; loading the capsule onto a flow cell or nanochannel; releasing the cellular contents such as nucleic acids from the capsule; and assaying the cellular contents such as sequencing the cell nucleic acids.

Processing in some cases comprises subjecting the cell nucleic acids to nucleic acid amplification in the aqueous core. Notably, processing or preparation does not in some cases comprise addition of a barcode to commonly identify the cell nucleic acids, or does not comprise addition of a barcode to commonly identify the cell nucleic acids.

A number of subdivision containment approaches are consistent with the disclosure herein. A solid capsule for example, may comprise a hydrogel shell, such as a porous shell. Alternately, subdivisions may be harbored in emulsion droplets or in wells, such as wells having porous hydrogel walls.

Cell lysis may comprise addition of an enzyme, such as a protease or a lipase. Capsule lysis may comprise addition of an enzyme such as a hydrogel degrading enzyme.

Lysis may also be effected under biological conditions such as those disclosed herein. Lysis, particularly of emulsion droplets or capsules such as hydrogel capsules, may comprise addition of a surfactant or addition of a solvent.

Sample preparation variously comprises reverse transcription, ligation, nucleic acid circularization, adapter addition, bridge amplification end addition or other modification. Sequencing library preparation may comprise nanopore sequencing library preparation or bridge amplification preparation, among others.

Loading a sample subdivision variously comprises delivering the capsule to a well on the flow cell, such as a well is sized to accommodate no more than one capsule. Alternately, loading comprises delivering the capsule to a bounded reaction site on the flow cell.

Releasing subdivision contents variously comprises degrading the capsule under physiological conditions such as those disclosed elsewhere herein. Releasing may occur at a pH within a range from 5-9, at a temperature within a range from 4-70 degrees Celsius, and may comprise mechanical, chemical or enzymatic degradation, among other approaches disclosed herein.

Molecular analysis may comprise sequencing such as nanopore sequencing, sequencing by extension such as sequencing by synthesis or sequencing by binding, or rolling circle long read sequencing, among others.

Also disclosed are sample libraries such as nucleic acid libraries and molecule measurement datasets such as nucleic acid datasets produced through practice of any of the above-mentioned methods.

Consistent with the above, disclosed herein are subdivision populations wherein subdivisions each comprise contents of a single cell, wherein the subdivisions are physically separate, wherein the contents are held distinctly from one another in hydrogel capsules, wherein small molecules are able to traffic among the hydrogel capsules, and wherein the hydrogel capsules in some cases do not comprise capsule-identifying or unique barcode labels.

Also disclosed herein are nucleic acid datasets comprising a plurality of nucleic acid sequence sets, wherein separate nucleic acid sequence sets of the plurality of nucleic acid sequence sets are distinguished by non-nucleic acid positional tags indicative of a common position where nucleic acids of separate nucleic acid sequence sets were sequenced, and wherein the separate nucleic acid sequence sets do not differ in exogenously added set-identifying nucleic acid barcode sequences.

Similarly, disclosed herein are nucleic acid datasets comprising sample nucleic acid sequence of a first nucleic acid sequence set arising from a first subdivision and a second nucleic acid sequence set arising from a second subdivision, wherein the first nucleic acid sequence set comprises nucleic acids associated with a first sequencing location and wherein the second nucleic acid sequence set comprises nucleic acids associated with a second sequencing location, and wherein the first nucleic acid sequence set and the second nucleic acid sequence set are not distinguished by set-identifying exogenously added nucleic acid tags.

In some such datasets, the first nucleic acid sequence set and the second nucleic acid sequence set comprise nucleic acid molecule sequences that are mutually indistinguishable, or are 100% identical.

Alternately, in some cases the first nucleic acid sequence set and the second nucleic acid sequence set differ in relative abundance of an identical nucleic acid.

A feature of a number of these datasets is that copies of the identical nucleic acid are not tagged by nucleic acid set identifying barcodes.

Often, the first subdivision comprises a first cell and the second subdivision comprises a second cell, though non-cell subdivision contents are also contemplated herein.

Returning to the example of cells, the first cell and the second cell arise from a common tissue in some cases. The first cell and the second cell may differ by a genetic mutation relative to one another.

In some datasets herein, at least 90% of the sequence arises from sample nucleic acids, or at least 95% of the sequence arises from sample nucleic acids, or at least 99% of the sequence arises from sample nucleic acids.

In some cases, at least one molecule sequence consists of sample nucleic acid sequence and library generation nucleic acid, and does not comprise exogenously added identifying sequence, or at least 90% of molecule sequence consists of sample nucleic acid sequence and library generation nucleic acid, and does not comprise exogenously added identifying sequence, or least 95% of molecule sequence consists of sample nucleic acid sequence and library generation nucleic acid, and does not comprise exogenously added identifying sequence, or at least 99% of molecule sequence consists of sample nucleic acid sequence and library generation nucleic acid, and does not comprise exogenously added identifying sequence.

Also disclosed herein are nucleic acid libraries from which some or all of the nucleic acid datasets may be generated.

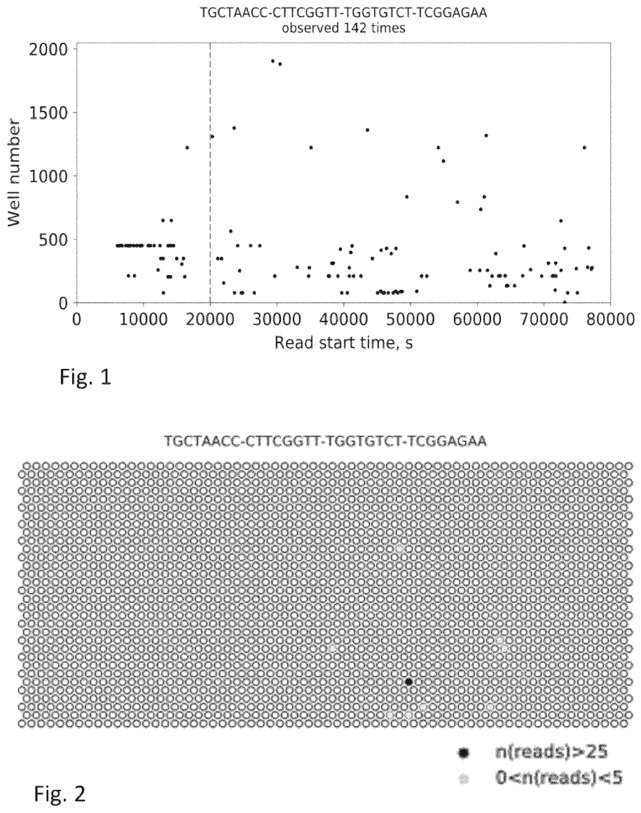

Turning to the Figures, one sees the following. At FIG. 1, one sees well distribution as a function of read start time for 142 observations of the barcode TGCTAACC-CTTCGGTT-TGGTGTTCT-TCGGAGAA. The x-axis is in seconds, ranging from 0-80,000 seconds in 10,000 second intervals. The y-axis is well number for a MinION flow cell. A dashed line indicates the 20,000 second time point.

This figure demonstrates that, among early reads, the significant majority arose from a common well, while over time the well locations from which barcoded reads arose became more dispersed. This indicates that microcapsule release was localized to an individual well, and that over time individual molecules diffused from the initial well and became dispersed across the array over time.

FIG. 2 presents the positional distribution of reads arising from the first 20,000 seconds of the sequencing run presented in FIG. 1. One sees that the majority of the reads localize to a single well, while a few additional reads amount to less than 5 reads each. Many of these additional wells are near to the single dominant well.

### Numbered Embodiments

The disclosure is further understood in light of the following partial list of numbered embodiments. 1. A method of delivering sample subdivisions to discrete positions on an assay region, comprising subdividing a sample into encapsulated subdivision capsules, delivering the encapsulated subdivision capsules to the assay region, and releasing contents of the encapsulated subdivision capsules at the assay region. 2. The method of any embodiment herein such as number 1, wherein releasing the contents is effected such that at least 10% of a given capsule content occupies a subregion of the assay region in which at least 50% of the sample content originates from the given capsule, and this applies to the content from at least 1 capsule. 3. The method of any embodiment herein such as number 1, wherein releasing the contents is effected such that at least 50% of the contents of the encapsulated subdivision capsules do not intermingle. 4. The method of any embodiment herein such as number 1, wherein releasing the contents is effected such that at least 75% of the contents of the encapsulated subdivision capsules do not intermingle. 5. The method of any embodiment herein such as number 1, wherein releasing the contents is effected such that at least 90% of the contents of the encapsulated subdivision capsules do not intermingle. 6. The method of any embodiment herein such as number 1, wherein releasing the contents is effected such that at least 99% of the contents of the encapsulated subdivision capsules do not intermingle. 7. The method of any embodiment herein such as number 1, wherein at least 60% of the contents of the encapsulated subdivision capsules do not intermingle. 8. The method of any embodiment herein such as number 1, wherein at least 70% of the contents of the encapsulated subdivision capsules do not intermingle. 9. The method of any embodiment herein such as number 1, wherein at least 80% of the contents of the encapsulated subdivision capsules do not intermingle. 10. The method of any embodiment herein such as number 1, wherein at least 90% of the contents of the encapsulated subdivision capsules do not intermingle. 11. The method of any embodiment herein such as number 1, wherein at least 95% of the contents of the encapsulated subdivision capsules do not intermingle. 12. The method of any embodiment herein such as number 1, wherein the sample comprises nucleic acids. 13. The method of any embodiment herein such as number 1, wherein the sample comprises cells. 14. The method of any embodiment herein such as number 1, wherein the sample comprises polypeptides. 15. The method of any embodiment herein such as number 1, wherein the assay region comprises a flow cell. 16. The method of any embodiment herein such as number 1, wherein the assay region comprises a microchannel. 17. The method of any embodiment herein such as number 1, wherein the assay region comprises a plurality of microchannels. 18. The method of any embodiment herein such as number 1, wherein the assay region comprises a cell culture medium. 19. The method of any embodiment herein such as number 1, wherein releasing contents of the encapsulated subdivision capsules distinctly comprises releasing contents of the encapsulated subdivision capsules at distinct locations of the assay region. 20. The method of any embodiment herein such as number 19, wherein the distinct locations comprise microchannel wells. 21. The method of any embodiment herein such as number 19, wherein the distinct locations comprise microchannel wells comprise walled off areas. 22. The method of any embodiment herein such as number 1, wherein releasing contents of the encapsulated subdivision capsules distinctly comprises releasing contents of the encapsulated subdivision capsules at temporally distinct times. 23. The method of any embodiment herein such as number 1, comprising assaying polypeptides of a first sample subdivision and a second sample subdivision of the encapsulated sample subdivisions, correlating assay results of the first sample subdivision to a first sample subdivision position and correlating sequences of the second sample subdivision to a second sample subdivision position. 24. The method of any embodiment herein such as number 1, comprising sequencing nucleic acids of a first sample subdivision and a second sample subdivision of the encapsulated sample subdivisions, correlating sequences of the first sample subdivision to a first sample subdivision position and correlating sequences of the second sample subdivision to a second sample subdivision position. 25. The method of any embodiment herein such as number 24, wherein the sequencing comprises nanopore sequencing. 26. The method of any embodiment herein such as number 24, wherein the sequencing comprises sequencing by extension. 27. The method of any embodiment herein such as number 24, wherein the sequencing comprises sequencing by synthesis. 28. The method of any embodiment herein such as number 24, wherein the sequencing comprises long read sequencing. 29. The method of any embodiment herein such as number 24, wherein the distinct locations comprise wells. 30. The method of any embodiment herein such as number 24, wherein the distinct locations comprise nanopore openings. 31. The method of any embodiment herein such as number 24, wherein the distinct locations comprise flow cell regions. 32. The method of any embodiment herein such as number 24, comprising assigning sequences correlated to the first sample subdivision position to a first encapsulated sample subdivision, and assigning sequences correlated to the second sample subdivision to a second encapsulated sample subdivision, such that sequences are assigned to encapsulated sample subdivisions without relying upon exogenously added tags. 33. The method of any embodiment herein such as numbers 1-32, wherein the sample is subjected to at least one reaction prior to the releasing. 34. The method of any embodiment herein such as number 33, wherein the at least one reaction comprises cell lysis. 35. The method of any embodiment herein such as number 33, wherein the at least one reaction comprises nucleic acid amplification. 36. The method of any embodiment herein such as number 33, wherein the at least one reaction does not comprise capsule-specifying sample barcoding. 37. The method of any embodiment herein such as number 33, wherein the capsules do not comprise capsule-specifying barcodes. 38. The method of any embodiment herein such as number 33, wherein the encapsulated sample subdivisions do not comprise capsule-specifying barcodes. 39. The method of any embodiment herein such as number 33, wherein the capsules comprise a porous shell and an aqueous core, and wherein the sample subdivisions reside in the aqueous core. 40. The method of any embodiment herein such as number 39, wherein the solid shell comprises a hydrogel. 41. The method of any embodiment herein such as numbers 1-38, wherein the releasing comprises degrading the capsules under physiological conditions. 42. The method of any embodiment herein such as numbers 1-38, wherein the releasing occurs at a pH within a range from 5-9. 43. The method of any embodiment herein such as numbers 1-38, wherein the releasing occurs at a temperature within a range from 4-50 degrees Celsius. 44. The method of any embodiment herein such as numbers 1-38, wherein the releasing comprises enzymatic digestion. 45. The method of any embodiment herein such as numbers 1-44, wherein the sample comprises a plurality of cells. 46. The method of any embodiment herein such as number 45, wherein the plurality of cells are drawn from a common source. 47. A method of sorting sample assay results, comprising subdividing a sample to a first sample set and a second sample set, assaying the first sample set at the first location so as to associate a first location identifier to sequences of the first sample set, assaying the second sample set at the second sample location so as to associate a second location identifier to assay results of the second sample set, associating assay results having the first assay identifier to the first sample set, and associating assay results having the second sequence identifier to the second sample set. 48. The method of any embodiment herein such as number 47, wherein the sample assay results comprise nucleic acid sequence, and wherein the sample comprises nucleic acids. 49. The method of any embodiment herein such as number 47, wherein the sample assay results comprise protein information. 50. The method of any embodiment herein such as number 47, wherein the first location and the second location are spatially separated. 51. The method of any embodiment herein such as number 47, wherein the first location and the second location are temporally separated. 52. The method of any embodiment herein such as number 47, wherein the first location and the second location are spatially separated and temporally separated. 53. The method of any embodiment herein such as number 48, wherein the first location identifier and the second location identifier do not differ by sequence derived from exogenously added sample set identifying nucleic acid oligos. 54. The method of any embodiment herein such as number 48, wherein the first location and the second location are on a common flow cell. 55. The method of any embodiment herein such as number 48, wherein sequencing the first sample set and sequencing the second sample set occur concurrently. 56. The method of any embodiment herein such as number 48, wherein sequencing the first sample set and sequencing the second sample set occur as part of a common sequencing run. 57. The method of any embodiment herein such as number 48, wherein the sequencing comprises nanopore sequencing. 58. The method of any embodiment herein such as number 48, wherein the sequencing comprises sequencing by extension. 59. The method of any embodiment herein such as number 48, wherein the distinct locations comprise wells. 60. The method of any embodiment herein such as numbers 47-59, wherein subdividing a sample comprises encapsulating the first sample set in a first capsule and the second sample set in a second capsule. 61. The method of any embodiment herein such as number 60, wherein the first capsule comprises an outer shell and an aqueous core, and wherein the first sample set resides in the aqueous core. 62. The method of any embodiment herein such as number 61, wherein the outer shell is porous. 63. The method of any embodiment herein such as numbers 60-62, wherein subdividing comprises delivering the first capsule to the first location, and releasing the first sample set from the first capsule. 64. The method of any embodiment herein such as number 63, wherein releasing the first sample set comprises degrading the outer shell under physiological conditions. 65. The method of any embodiment herein such as number 64, wherein the releasing occurs at a pH within a range from 5-9. 66. The method of any embodiment herein such as number 64, wherein the releasing occurs at a temperature within a range from 4-50 degrees Celsius. 67. The method of any embodiment herein such as number 64, wherein the releasing comprises enzymatic digestion. 68. The method of any embodiment herein such as numbers 47-67, wherein the first sample set comprises a first cell, and the second sample set comprises a second cell. 69. The method of any embodiment herein such as numbers 47-67, wherein the first location identifier comprises information identifying the first location as origin of a target sequence arising from the first sample set. 70. The method of any embodiment herein such as numbers 47-67, wherein the second location identifier comprises information identifying the second location as origin of a target sequence arising from the second sample set. 71. A method of mapping nucleic acids to a single cell, comprising encapsulating a cell into a solid capsule having an aqueous core; subjecting the cell to lysis to yield cell nucleic acids in the aqueous core; subjecting the cell nucleic acids to sequencing library preparation; loading the capsule onto a flow cell; releasing the cell nucleic acids from the capsule; and sequencing the cell nucleic acids, wherein locally proximal sequence reads are assigned to a single cell. 72. The method of any embodiment herein such as number 71, wherein locally proximal sequence reads arise from a common microchannel. 73. The method of any embodiment herein such as number 71, wherein locally proximal sequence reads arise from a common fenced in region. 74. The method of any embodiment herein such as number 71, wherein locally proximal sequence reads arise from an adjacent microchannel. 75. The method of any embodiment herein such as number 71, wherein locally proximal sequence reads arise from an adjacent fenced in region. 76. The method of any embodiment herein such as number 71, wherein locally proximal sequence reads arise from a region harboring reads at a density above a threshold. 77. The method of any embodiment herein such as number 71, wherein locally proximal sequence reads are identified as being no more than a set distance from a local density maximum. 78. The method of any embodiment herein such as number 77, wherein the set distance is within 10 um. 79. The method of any embodiment herein such as number 77, wherein the set distance is within 50 um. 80. The method of any embodiment herein such as number 77, wherein the set distance is within 100 um. 81. The method of any embodiment herein such as number 71, wherein the locally proximal sequence reads are temporally proximal. 82. The method of any embodiment herein such as number 71, wherein locally proximal sequence reads are temporally adjacent. 83. The method of any embodiment herein such as number 71, comprising subjecting the cell nucleic acids to nucleic acid amplification in the aqueous core. 84. The method of any embodiment herein such as number 71, wherein the sequencing library preparation does not comprise addition of a barcode to commonly identify the cell nucleic acids. 85. The method of any embodiment herein such as number 71, wherein the method does not comprise addition of a barcode to commonly identify the cell nucleic acids. 86. The method of any embodiment herein such as number 71, wherein the solid capsule comprises a hydrogel shell. 87. The method of any embodiment herein such as number 71, wherein the solid capsule comprises a porous shell. 88. The method of any embodiment herein such as number 71, wherein the lysis comprises addition of an enzyme. 89. The method of any embodiment herein such as number 88, wherein the enzyme comprises a protease. 90. The method of any embodiment herein such as number 88, wherein the enzyme comprises a lipase. 91. The method of any embodiment herein such as number 71, wherein the lysis comprises alteration of pH. 92. The method of any embodiment herein such as number 71, wherein the lysis comprises heat. 93. The method of any embodiment herein such as number 71, wherein the lysis comprises addition of a surfactant. 94. The method of any embodiment herein such as number 71, wherein the lysis comprises addition of a solvent. 95. The method of any embodiment herein such as number 71, wherein the sequencing library preparation comprises reverse transcription. 96. The method of any embodiment herein such as number 71, wherein the sequencing library preparation comprises amplification. 97. The method of any embodiment herein such as number 96, wherein the amplification comprises at least one amplification selected from the list consisting of polymerase chain reaction amplification, multiple displacement amplification, in vitro transcription, and rolling circle amplification. 98. The method of any embodiment herein such as number 71, wherein the sequencing library preparation comprises circularization. 99. The method of any embodiment herein such as number 71, wherein the sequencing library preparation comprises adapter addition. 100. The method of any embodiment herein such as number 71, wherein the sequencing library preparation comprises nanopore sequencing library preparation. 101. The method of any embodiment herein such as number 71, wherein the loading comprises delivering the capsule to a well on the flow cell. 102. The method of any embodiment herein such as number 64, wherein the well is sized to accommodate no more than one capsule. 103. The method of any embodiment herein such as number 71, wherein the loading comprises delivering the capsule to a bounded reaction site on the flow cell. 104. The method of any embodiment herein such as number 71, wherein the loading comprises delivering the capsule to an unstructured flowcell. 105. The method of any embodiment herein such as number 71, wherein the loading comprises delivering the capsule to a flowcell that does not have wells. 106. The method of any embodiment herein such as number 71, wherein the loading comprises delivering the capsule to a flow cell that does not have fences. 107. The method of any embodiment herein such as number 71, wherein the loading comprises delivering the capsule to an unpatterned flowcell. 108. The method of any embodiment herein such as number 71, wherein the releasing comprises degrading the capsule physiological conditions. 109. The method of any embodiment herein such as number 71, wherein the releasing occurs at a pH within a range from 5-9. 110. The method of any embodiment herein such as number 71, wherein the releasing occurs at a temperature within a range from 4-50 degrees Celsius. 111. The method of any embodiment herein such as number 71, wherein the releasing comprises enzymatic digestion. 112. The method of any embodiment herein such as number 71, wherein the sequencing comprises nanopore sequencing. 113. The method of any embodiment herein such as number 71, wherein the sequencing comprises sequencing by extension. 114. The method of any embodiment herein such as number 71, wherein the sequencing comprises long read sequencing. 115. A nucleic acid dataset comprising nucleic acid sequences generated by the method of any embodiment herein such as numbers 1-46. 116. A nucleic acid dataset comprising nucleic acid sequences generated by the method of any embodiment herein such as numbers 47-68. 117. A nucleic acid dataset comprising nucleic acid sequences generated by the method of any embodiment herein such as numbers 71-114. 118. A nucleic acid dataset comprising a plurality of nucleic acid sequence sets, wherein separate nucleic acid sequence sets of the plurality of nucleic acid sequence sets are distinguished by non-nucleic acid positional or temporal tags indicative of a common locational or temporal position where nucleic acids of separate nucleic acid sequence sets were sequenced, and wherein the separate nucleic acid sequence sets do not differ in exogenously added set-identifying nucleic acid barcode sequences. 119. A nucleic acid dataset comprising sample nucleic acid sequence of a first nucleic acid sequence set arising from a first subdivision and a second nucleic acid sequence set arising from a second subdivision, wherein the first nucleic acid sequence set comprises nucleic acids associated with a first sequencing location and wherein the second nucleic acid sequence set comprises nucleic acids associated with a second sequencing location, and wherein the first nucleic acid sequence set and the second nucleic acid sequence set are not distinguished by set-identifying exogenously added nucleic acid tags. 120. The nucleic acid dataset of any embodiment herein such as number 119, wherein the first nucleic acid sequence set and the second nucleic acid sequence set comprise nucleic acid molecule sequences that are mutually indistinguishable. 121. The nucleic acid dataset of any embodiment herein such as number 119, wherein the first nucleic acid sequence set and the second nucleic acid sequence set comprise nucleic acid molecule sequences that are 100% identical. 122. The nucleic acid dataset of any embodiment herein such as numbers 119-121, wherein the first nucleic acid sequence set and the second nucleic acid sequence set differ in relative abundance of an identical nucleic acid. 123. The nucleic acid dataset of any embodiment herein such as number 122, wherein copies of the identical nucleic acid are not tagged by nucleic acid set identifying barcodes. 124. The nucleic acid dataset of any embodiment herein such as numbers 119-123, wherein the first subdivision comprises a first cell and the second subdivision comprises a second cell. 125. The nucleic acid dataset of any embodiment herein such as number 124, wherein the first cell and the second cell arise from a common tissue. 126. The nucleic acid dataset of any embodiment herein such as number 124, wherein the first cell and the second cell differ by a genetic mutation relative to one another. 127. The nucleic acid dataset of any embodiment herein such as numbers 119-126, wherein at least 90% of the sequence arises from sample nucleic acids. 128. The nucleic acid dataset of any embodiment herein such as numbers 119-126, wherein at least 95% of the sequence arises from sample nucleic acids. 129. The nucleic acid dataset of any embodiment herein such as numbers 119-126, wherein at least 99% of the sequence arises from sample nucleic acids. 130. The nucleic acid dataset of any embodiment herein such as numbers 119-126, wherein at least one molecule sequence consists of sample nucleic acid sequence and library generation nucleic acid, and does not comprise exogenously added identifying sequence. 131. The nucleic acid dataset of any embodiment herein such as numbers 119-126, wherein at least 90% of molecule sequence consists of sample nucleic acid sequence and library generation nucleic acid, and does not comprise exogenously added identifying sequence. 132. The nucleic acid dataset of any embodiment herein such as numbers 119-126, wherein at least 95% of molecule sequence consists of sample nucleic acid sequence and library generation nucleic acid, and does not comprise exogenously added identifying sequence. 133. The nucleic acid dataset of any embodiment herein such as numbers 119-126, wherein at least 99% of molecule sequence consists of sample nucleic acid sequence and library generation nucleic acid, and does not comprise exogenously added identifying sequence. 134. A method of delivering sample subdivisions to discrete positions on an assay region, comprising subdividing a sample into encapsulated subdivision capsules, delivering the encapsulated subdivision capsules to the assay region, and releasing contents of the encapsulated subdivision capsules at the assay region such that at least 50% of the contents of the encapsulated subdivision capsules do not intermingle. 135. A method of delivering sample subdivisions to discrete positions on an assay region, comprising subdividing a sample into encapsulated subdivision capsules, delivering the encapsulated subdivision capsules to the assay region, and releasing contents of the encapsulated subdivision capsules at the assay region such that at least 10% of a given capsule content occupies a subregion of the assay region in which at least 50% of the sample content originates from the given capsule, and this applies to the content from at least 1 capsule.

As used herein, reads from distinct microcapsules “intermingle” when a nearest adjacent read to a read of a first microcapsule arises from a nucleic acid of a second microcapsule.

As used herein, the term “about” in the context of a number represents a range spanning from 10% less than that number to 10% greater than that number. The term “about” in the context of a range represents a larger range spanning from 10% less than the lower limit of the initial range to 10% greater than the upper limit of the initial range.

As used herein, the phrase “at least one selected from the list consisting of” A, B, and C refers to a set comprising either A only, A and B, A and C, B and C, or A, B, and C, and optionally other constituents.

### EXAMPLES

Example 1. One-pore-one-cell sequencing. A cell is encapsulated into a porous hydrogel capsule. The capsule is permeable to small molecule reagents but retains nucleic acid macromolecules of about 500 bp or larger and well as comparably sized proteins.

By changing the aqueous environment of the capsule, one can manipulate the internal environment of the capsule.

The capsule is successively exposed to environmental conditions so as to create conditions within the porous capsule conducive to cell lysis, protein degradation, RNA degradation, and nucleic acid processing preparatory of nanopore sequencing. The cell lysate is not subjected to pipetting pursuant to this process, such that the DNA in the lysate is unsheared and relatively intact as full length or near full length molecules.

The capsule is deposited at a well having a nanopore at its base. The capsule is lysed using a hydrogel degrading enzyme, such that the processed cell lysate is released into the well.

The processed cell lysate passes through the nanopore. Various processed nucleic acid molecules pass through the nanopore in succession, and their sequences are determined.

The sequence data is generated by the nanopore in closely associated temporal succession, such that the cell lysate nucleic acid sequences are identifiable as arising from a common cell in a common capsule by the nanopore which generated their sequence and by the temporal proximity of the sequence data created. Accordingly, although the nucleic acids of the cell lysate do not share commonly identifying exogenously added barcodes, one may nonetheless assign them to a common cell of origin.

Once the sequencing run is completed, a second capsule comprising processed cell lysate from a second cell is deposited into the well. The capsule is similarly lysed and the processed cell lysate of the second cell passes through the nanopore. Various processed nucleic acid molecules of the second cell pass through the nanopore in succession, and their sequences are determined.

The sequence data from the second cell lysate is generated by the nanopore in closely associated temporal succession, such that the second cell lysate nucleic acid sequences are identifiable as arising from a common second cell in a common capsule by the nanopore which generated their sequence and by the temporal proximity of the sequence data created. Accordingly, although the nucleic acids of the second cell lysate do not share commonly identifying or cell-distinguishing exogenously added barcodes, one may nonetheless assign them to the second common cell of origin, and can distinguish them from the similarly unbarcoded nucleic acid sequences of the first cell lysate.

Example 2. Positional cell identification. A population of cells is encapsulated into a population of porous hydrogel capsules such that no single capsule harbors more than one cell. The capsules are permeable to small molecule reagents but retain nucleic acid macromolecules of about 500 bp or larger and well as comparably sized proteins.

By changing the aqueous environment of the capsules, one can manipulate the internal environment of the capsules.

The capsule population is successively exposed to environmental conditions so as to create conditions within the porous capsule conducive to cell lysis, protein degradation, DNA degradation, RNA reverse transcription and nucleic acid processing preparatory of colony formation and sequencing by synthesis. The cell lysate is not subjected to pipetting pursuant to this process, such that the RNA and reverse transcribed DNA in the lysate is unsheared and relatively intact as full length or near full length molecules.

The capsule population is deposited on the surface of a flow cell configured for colony formation and sequencing by synthesis. The capsule population density is selected such that capsules are not uniformly, locally packed. That is, there is often space between capsules on the flow cell surface.

Capsules are lysed and lysate nucleic acid contents are bound to the flow cell local environment. In some cases lysis is performed in a carrier having a viscosity greater than that of water, so as to reduce capsule contents diffusion prior to deposition. Nucleic acids are subjected to colony formation and sequencing by synthesis.

Nucleic acid sequences are obtained from the colonies on the flow cell. Locally proximal colony signals are assigned to a common cell of origin, without relying upon cell-identifying or capsule-identifying exogenously added oligo barcode sequences. Accordingly, nucleic acid read information is grouped by cell or sample subdivision origin, and whole genome or transcriptome-level conclusions are made as to transcript abundance levels and accumulation level correlations in specific cells of the cell populations.

As the nucleic acids do not harbor exogenously added cell-or capsule-identifying barcode sequence, substantially more of each read represents cell lysate sequence, and proportionately more cell lysate sequence is obtained. Consequently, proportionately fewer reads are required to obtain a comparable amount of cell sequence.

Example 3. Mapping of released nucleic acids. E. coli cells were encapsulated into DexMAB microcapsules, lysed internally, and their DNA was proceeded into a barcoded library for MinION sequencing. The MinION Nanopore flow cell bears 2048 pores at the bottom of ~100 um diameter wells positioned to form a honeycomb-like hexagonal pattern. Nanopore sequencing library preparation on microcapsule-contained DNA was performed using the Genomic DNA by Ligation (SQK-LSK112) kit by Oxford Nanopore. The protocol was modified to replace nucleic acid purification steps using beads or columns with microcapsule washes by pelleting the capsules and replacing the supernatant. Nucleic acids were barcoded with microcapsule specific barcodes.

The sample loaded on the MinION R9.4.1 flow cell consisted of a capsule suspension rather than a homogenous solution. Loading beads were not used. 1 ul of dextranase was added directly to the flow cell to release capsule content. Sequencing was performed for 22 hours. Sequence reads harboring barcodes were subjected to further analysis. No measures to limit diffusion between wells, such as covering the well array with mineral oil, were taken in this experiment. Therefore, over time, diffusion is expected to lead to multiple pore usage.

In line with the expectation, reads bearing the most abundant barcode were predominantly originating from the same pore (or well) at the beginning of the sequencing run with more diverse pore usage over time (FIG. 1). Out of a total of 42 barcode-bearing reads obtained in the first 20,000 seconds of the sequencing run, 26 originated from a single pore.

Results are shown in FIG. 1 and FIG. 2. At FIG. 1, one sees MinION flow cell channel usage by most abundant barcode over time. The dashed gray line marks a suggested read start time threshold (20000 s or 5.55 h) below which DNA fragment diffusion across the flow cell has not yet occurred.

As shown in FIG. 2, channels form a 2-dimensional array. Pores of origin of reads bearing the most abundant barcode in the first 20000 seconds (5.55 h) of the sequencing run are indicated by shading, with the pore exhibiting 26 reads shaded black, while eight additional pores exhibiting 1-5 reads of the same barcode are shaded light grey. Circles show the physical xy layout of the MinION R9.4.1 flow cell used in the experiment. The dot filled in black marks the pore from which 26 reads originated. Note, that the difference in channel number between two reads is generally not indicative of physical proximity because the channels are numbered in a non-obvious way on the MinION flow cell.

These results clearly demonstrate that direct loading of capsules on a Nanopore flow cell allows single-microcapsule, and therefore single-cell, resolution using well coordinates to demultiplex reads.

Example 4. Concentrated contents sequencing. A environmental sample is encapsulated into microcapsules. Encapsulated cells are allowed to divide, and are lysed and packaged into libraries for sequencing. One microcapsule encapsulates a cell that occurs at a frequency of 0.1% of cells in the sample

Microcapsules are delivered onto a nanopore flow cell and the packaged library contents are released. Microcapsule contents diffuse, such that contents of a single microcapsule are not contained within a single pore well. Nonetheless, a given well that contains the microcapsule harboring the cell occurring at a frequency of 0.1% in the sample with a single-cell amplified genome (as opposed to an empty capsule) is enriched for reads from that rare cell. Even if as much as 90% of the microcapsule contents diffuse out, and as much as for example 50% of the reads from that well arise from nucleic acids that diffused into the well, the well is still substantially enriched for reads from the rare cell.

Reads are assembled into contigs, with the assembly process informed by well of origin. The reads from the rare-cell well assemble into two sets of contigs, one set corresponding to the rare cell and another set corresponding to nucleic acids that diffused into the well. The set comprising rare cell reads exhibits a substantially larger N50 or average contig size, while many of the diffused in reads assemble as singletons.

A second reaction is performed, using total DNA from an environmental sample without encapsulation. Reads corresponding to the rare cell represent about 0.1% of the total reads. In the absence of additional information such as well of origin information, reads from the rare cell genome are frequently misassembled with reads from other organisms.

This example illustrates the value of well-of-origin information when samples are delivered via microcapsules that result in wells or flow cell locations being enriched for specific microcapsule contents, even when diffusion occurs.

We claim:

1.-70. (canceled)

71. A method of mapping nucleic acids to a single cell, comprising encapsulating a cell into a solid capsule having an aqueous core; subjecting the cell to lysis to yield cell nucleic acids in the aqueous core; subjecting the cell nucleic acids to sequencing library preparation; loading the capsule onto a flow cell or microchannel array; releasing the cell nucleic acids from the capsule; and sequencing the cell nucleic acids, wherein locally proximal sequence reads are assigned to a single cell.

72. The method of claim 71, wherein locally proximal sequence reads arise from a common microchannel.

73. The method of claim 71, wherein locally proximal sequence reads arise from a common fenced in region.

74. The method of claim 71, wherein locally proximal sequence reads arise from an adjacent microchannel.

75. The method of claim 71, wherein locally proximal sequence reads arise from an adjacent fenced in region.

76. (canceled)

77. The method of claim 71, wherein locally proximal sequence reads are identified as being no more than a set distance from a local density maximum.

78. The method of claim 77, wherein the set distance is within 10 um.

79.-81. (canceled)

82. The method of claim 71, wherein locally proximal sequence reads are temporally adjacent.

83. The method of claim 71, comprising subjecting the cell nucleic acids to nucleic acid amplification in the aqueous core.

84. The method of claim 71, wherein the sequencing library preparation does not comprise addition of a barcode to commonly identify the cell nucleic acids.

85. The method of claim 71, wherein the method does not comprise addition of a barcode to commonly identify the cell nucleic acids.

86.-94. (canceled)

95. The method of claim 71, wherein the sequencing library preparation comprises reverse transcription.

96. The method of claim 71, wherein the sequencing library preparation comprises amplification.

97. The method of claim 96, wherein the amplification comprises at least one amplification selected from the list consisting of polymerase chain reaction amplification, multiple displacement amplification, in vitro transcription, and rolling circle amplification.

98. The method of claim 71, wherein the sequencing library preparation comprises circularization.

99. The method of claim 71, wherein the sequencing library preparation comprises adapter addition.

100. The method of claim 71, wherein the sequencing library preparation comprises nanopore sequencing library preparation.

101.-107. (canceled)

108. The method of claim 71, wherein the releasing comprises degrading the capsule under physiological conditions.

109.-110. (canceled)

111. The method of claim 71, wherein the releasing comprises enzymatic digestion.

112. The method of claim 71, wherein the sequencing comprises nanopore sequencing.

113. The method of claim 71, wherein the sequencing comprises sequencing by extension.

114. The method of claim 71, wherein the sequencing comprises long read sequencing.

115.-135. (canceled)
